(19) United States
(12) Patent Application Publication
(10) Pub. No.: US 2024/0066303 A1
(43) Pub. Date: Feb. 29, 2024

(54) Methods and Systems for Determining Baseline Voltages for Sensed Neural Response in an Implantable Stimulator Device System

(71) Applicant: Boston Scientific Neuromodulation Corporation, Valencia, CA (US)

(72) Inventors: Philip Weiss, Sherman Oaks, CA (US); Venugopal Allavatam, Saratoga, CA (US); Andrew Haddock, Los Angeles, CA (US); Adarsh Jayakumar, Valencia, CA (US); Joshua Uyeda, Los Angeles, CA (US)

(21) Appl. No.: 18/453,916

(22) Filed: Aug. 22, 2023

Related U.S. Application Data
(60) Provisional application No. 63/373,966, filed on Aug. 30, 2022.

(51) Int. Cl. A61N 1/36; A61N 1/05; A61N 1/372

(52) U.S. Cl. CPC A61N 1/36139; A61N 1/0534; A61N 1/36125; A61N 1/372

(57) ABSTRACT

Techniques for determining baseline voltages to assess sensed neural responses or other sensed signals in an implantable stimulator device are disclosed, which allows features of the neural responses or other signals to be more easily and reliably established. Features of the neural response, indicative of the AC characteristics of the responses, may be used to control or monitoring stimulation in the device, and certain features may vary with a DC offset voltage in the tissue. The determined baseline voltages compensate for such DC offset voltages, and therefore allow certain AC features of the neural response to be determined more accurately and meaningfully.

### CROSS REFERENCE TO RELATED APPLICATIONS

This is a non-provisional of U.S. Provisional Patent Application Ser. No. 63/373,966, filed Aug. 30, 2022, which is incorporated herein by reference, and to which priority is claimed.

### FIELD OF THE INVENTION

This application relates to Implantable Medical Devices (IMDs), and more specifically to circuitry to assist with sensing neural responses to stimulation in an implantable stimulator device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) or Deep Brain Stimulation (DBS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In a DBS application, the electrode leads are implanted in the brain through holes in the skull, and lead extension are used to connect the leads to the IPG which is typically implanted under the clavicle (collarbone). In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. SCS therapy can relieve symptoms such as chronic back pain, while DBS therapy can alleviate Parkinsonian symptoms such as tremor and rigidity. IPG 10 as described should be understood as including External Trial Stimulators (ETSs), which mimic operation of the IPG 10 during trials periods when leads have been implanted in the patient but the IPG 10 has not. See, e.g., U.S. Pat. No. 9,259,574 (disclosing an ETS).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30i), as shown in the example of FIG. 2. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation that treats a symptom (e.g., pain) to a patient.

In the example of FIG. 2, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude −I from the tissue. This is an example of bipolar stimulation, in which the lead includes one anode pole and one cathode pole. Note that more than one electrode on the lead may be selected to act as an anode electrode to form an anode pole at a given time, and more than one electrode may be selected to act as a cathode to form a cathode pole at a given time, as explained further in U.S. Pat. No. 10,881,859. Stimulation provided by the IPG 10 can also be monopolar. In monopolar stimulation, the lead is programmed with a single pole of a given polarity (e.g., a cathode pole), with the conductive case electrode Ec acting as a return (e.g., an anode pole). Again, more than one electrode on the lead may be active to form the pole during monopolar stimulation.

IPG 10 as mentioned includes stimulation circuitry 28 to form prescribed stimulation at a patient's tissue. FIG. 3 shows an example of stimulation circuitry 28, which includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38. The DC-blocking capacitors 38 act as a safety measure to prevent DC current injection into the patient, as could occur for example if there is a circuit fault in the stimulation circuitry 28. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2, FIG. 3 shows operation during the first phase 30a in which electrode E1 has been selected as an anode electrode to source current I to the tissue R and E2 has been selected as a cathode electrode to sink current from the tissue. Thus PDAC1 and NDAC2 are digitally programmed to produce the desired current, I, with the correct timing (e.g., in accordance with the prescribed frequency and pulse widths). During the second phase the direction of this current would be reversed by digitally programming NDAC1 and PDAC2 to produce current I. Other stimulation circuitries 28 can also be used in the IPG 10, including ones that includes switching matrices between the electrode nodes ei 39 and the N/PDACs. See, e.g., U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519, which are incorporated by reference in their entireties. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage VH that powers the stimulation circuitry, various measurement circuits, etc.

Referring again to FIG. 2, the stimulation pulses as shown are biphasic, with each pulse comprising a first phase 30a followed thereafter by a second phase 30b of opposite polarity. Biphasic pulses are useful to actively recover any charge (during the second phase 30b) that might be stored on capacitive elements in the electrode current paths (during the first phase 30a), such as on the DC-blocking capacitors 38 that are otherwise used as a safety measure to prevent DC current injection into the patient. Charge recovery using phases 30a and 30b is said to be “active” because the P/NDACs in stimulation circuitry 28 actively drive a current, in particular during the last phase 30b to recover charge stored after the first phase 30a. However, passive charge recovery can also be implemented using passive charge recovery switches PRi 41 as shown in FIG. 3. These switches 41 when selected via assertion of control signals <Xi> couple each electrode node ei to a passive recovery voltage Vpr established on bus 43. As explained in U.S. Pat. Nos. 10,716,937 and 10,792,491, this allows any stored charge to be recovered through the patient's tissue, R. Control signals <Xi> are usually asserted to cause passive charge recovery after each pulse (e.g., after each last phase 30b) during periods 30c shown in FIG. 2. This may be followed by a quiet period 30d during which no currents are present.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc. The external systems of FIG. 4 can also be used to program or update the firmware in the IPG 10.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

A stimulator device is disclosed, which may comprise: a plurality of electrode nodes, wherein each of the electrode nodes is associated with a different electrode configured to contact a patient's tissue; stimulation circuitry configured to provide stimulation to the patient's tissue via one or more first of the electrode nodes; sense amplifier circuitry configured to sense a response to the stimulation at one or more second of the electrode nodes; control circuitry configured to: determine a baseline voltage from the sensed response, and determine at least one feature of the response using the baseline voltage, wherein the at least one feature is indicative of an AC characteristic of the response.

In one example, the baseline voltage is indicative of a DC offset voltage of the response. In one example, the control circuitry is configured to determine the baseline voltage by assessing a shape of the response. In one example, the control circuitry is configured to determine the baseline voltage as a first or last voltage value in the response. In one example, the control circuitry is further configured to determine one or more peaks in the response, and wherein the control circuitry is configured to determine the baseline voltage relative to a voltage value of at least one of the peaks. In one example, the control circuitry is further configured to determine either or both of a maximum peak or minimum peak in the response, and wherein the control circuitry is configured to determine the baseline voltage relative to the voltage value of either or both of the maximum peak and the minimum peak. In one example, the control circuitry is configured to determine the baseline voltage between the voltage value of the maximum peak and the voltage value of the minimum peak. In one example, the control circuitry is further configured to determine a slope of the response, and wherein the control circuitry is configured to determine the baseline voltage relative to a voltage value corresponding to a maximum slope in the response. In one example, the control circuitry is further configured to determine a curvature of the response, and wherein the control circuitry is configured to determine the baseline voltage relative to a voltage value corresponding to a maximum curvature in the response. In one example, the control circuitry is further configured to determine one or more segments in the response, and wherein the control circuitry is configured to determine the baseline voltage using at least one of the segments. In one example, the control circuitry is further configured to determine a longest of the one or more segments, and wherein the control circuitry is configured to determine the baseline voltage relative to at least one voltage value in the longest segment. In one example, the control circuitry is configured to determine the baseline voltage relative to either or both of a start voltage value and an end voltage value of the longest segment. In one example, the control circuitry is configured to determine the baseline voltage at a voltage value that either maximizes or minimizes a value of the at least one feature. In one example, the response comprises a stimulation artifact which results from an electromagnetic field that forms in the tissue as a result of the stimulation, and/or a neural response evoked in the tissue in response to the stimulation. In one example, the stimulation circuitry is configured to provide the stimulation in a sequence of pulses. In one example, the sense amplifier circuitry is configured to sense a response for each pulse. In one example, the control circuitry is configured to determine a unique baseline voltage for each of the responses, and wherein the control circuitry is configured to determine the at least one feature of each response using its baseline voltage. In one example, the control circuitry is configured to determine a baseline voltage for a plurality of the responses, and wherein the at least one feature of the plurality of responses is determined using the baseline voltage. In one example, the control circuitry comprises an analog-to-digital converter configured to digitize the sensed response, and wherein the control circuitry is configured to determine the baseline voltage using the digitized sensed response. In one example, the stimulation comprises therapeutic stimulation tailored to treat a symptom of the patient. In one example, the first of the electrode nodes and the second of the electrode nodes are different from each other. In one example, at least one of the first of the electrode nodes and at least one of the second of the electrode nodes are the same.

A method is disclosed for operating a stimulator device comprising a plurality of electrode nodes, wherein each of the electrode nodes is associated with a different electrode configured to contact a patient's tissue. The method may comprise: providing stimulation to the patient's tissue via one or more first of the electrode nodes; sensing a response to the stimulation at one or more second of the electrode nodes; determining a baseline voltage from the sensed response; and determining at least one feature of the response using the baseline voltage, wherein the at least one feature is indicative of an AC characteristic of the response.

In one example, the baseline voltage is indicative of a DC offset voltage of the response. In one example, the baseline voltage is determined by assessing a shape of the response. In one example, the baseline voltage is determined as a first or last voltage value in the response. In one example, the method further comprises determining one or more peaks in the response, wherein the baseline voltage is determined relative to a voltage value of at least one of the peaks. In one example, the method further comprises determining either or both of a maximum peak or minimum peak in the response, wherein the baseline voltage is determined relative to the voltage value of either or both of the maximum peak and the minimum peak. In one example, the baseline voltage is determined between the voltage value of the maximum peak and the voltage value of the minimum peak. In one example, the method further comprises determining a slope of the response, wherein the baseline voltage is determined relative to a voltage value corresponding to a maximum slope in the response. In one example, the method further comprises determining a curvature of the response, wherein the baseline voltage is determined relative to a voltage value corresponding to a maximum curvature in the response. In one example, the method further comprises determining one or more segments in the response, wherein the baseline voltage is determined using at least one of the segments. In one example, the method further comprising determining a longest of the one or more segments, wherein the baseline voltage is determined relative to at least one voltage value in the longest segment. In one example, the baseline voltage is determined relative to either or both of a start voltage value and an end voltage value of the longest segment. In one example, the baseline voltage is determined at a voltage value that either maximizes or minimizes a value of the at least one feature. In one example, the response comprises a stimulation artifact which results from an electromagnetic field that forms in the tissue as a result of the stimulation. In one example, the response comprises a neural response evoked in the tissue in response to the stimulation. In one example, the stimulation is provided in a sequence of pulses. In one example, a response to the stimulation is sensed for each pulse. In one example, a unique baseline voltage is determined for each of the responses, and wherein the at least one feature of each response is determined using its baseline voltage. In one example, the baseline voltage is determined for a plurality of the responses, and wherein the at least one feature of the plurality of responses is determined using the baseline voltage. In one example, the method further comprises digitizing the sensed response, wherein the baseline voltage is determined using the digitized sensed response. In one example, the stimulation comprises therapeutic stimulation tailored to treat a symptom of the patient. In one example, the first of the electrode nodes and the second of the electrode nodes are different from each other. In one example, at least one of the first of the electrode nodes and at least one of the second of the electrode nodes are the same.

A non-transitory computer readable medium is disclosed comprising instructions executable in a stimulator device comprising a plurality of electrode nodes, wherein each of the electrode nodes is associated with a different electrode configured to contact a patient's tissue, wherein the stimulator device is configured to provide stimulation to the patient's tissue via one or more first of the electrode nodes, wherein the instructions when executed are configured to cause the stimulator device to: sense a response to the stimulation at one or more second of the electrode nodes; determine a baseline voltage from the sensed response; and determine at least one feature of the response using the baseline voltage, wherein the at least one feature is indicative of an AC characteristic of the response.

### BRIEF DESCRIPTION OF THE DRAWINGS

FIG. 1 shows an Implantable Pulse Generator (IPG), in accordance with the prior art.

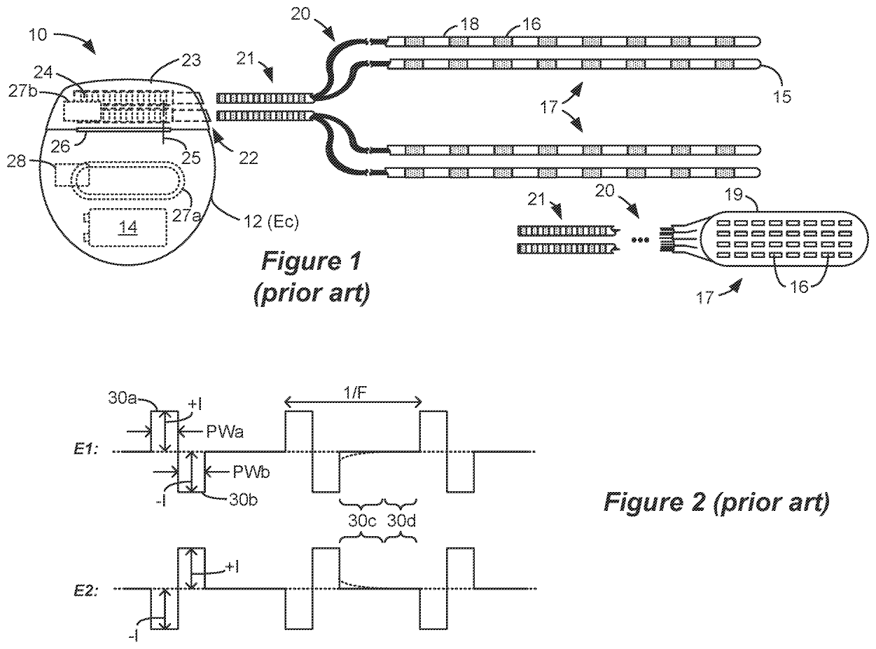

FIG. 2 shows an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows an IPG having the capability to sense an ESG signal, including a neural response and/or a stimulation artifact.

FIG. 6 shows stimulation producing an ESG signal, and the sensing of that signal at at least one electrode of the IPG.

FIGS. 7A and 7B respectively show an ESG signal and a neural response as sensed after they have been amplified and digitized.

FIG. 8A shows the influence of a DC offset voltage on a sensed ESG signal, and how the DC offset voltage can affect extraction of AC features of a neural response.

FIG. 8B shows how the determination of different baseline voltage to assess features in a neural response can the influence the determination of those features even when the DC offset voltage doesn't vary.

FIG. 9 shows an improved tissue signal detection algorithm, which includes a baseline determination algorithm to determine a baseline voltage that compensates for variation in the DC offset voltage.

FIGS. 10A-10G show different manners in which the baseline determination algorithm can operate to determine a baseline voltage by analyzing the shape of the sensed neural responses.

FIG. 11 shows another manner in which the baseline determination algorithm can operate to determine a baseline voltage by determining various segments in the sensed neural response.

FIG. 12 shows another manner in which the baseline determination algorithm can operate to determine a baseline voltage by determining a baseline voltage that maximizes or minimizes a value of a particular neural response feature.

FIG. 13 shows use by the baseline determination algorithm of a stimulation artifact in the sensed ESG signal to determine a baseline voltage for a sensed neural response.

FIG. 14 shows use by the baseline determination algorithm of a quiet period in the sensed ESG signal to determine a baseline voltage for a sensed neural response.

FIG. 15 shows use by the baseline determination algorithm of an ESG signal sensed in one timing channel to determine a baseline voltage for a neural response sensed in another channel.

FIG. 16 shows periodic determination by the baseline determination algorithm of baseline voltages for use in sensing neural responses.

### DETAILED DESCRIPTION

An increasingly interesting development in pulse generator systems is the addition of sensing capability to complement the stimulation that such systems provide. For example, and as explained in U.S. Patent Application Publication 2017/0296823, it can be beneficial to sense a neural response produced by neural tissue that has received stimulation from an IPG. The '823 Publication shows an example where sensing of neural responses is useful in an SCS context, and in particular discusses the sensing of Evoked Compound Action Potentials, or “ECAPs,” which comprise a cumulative response provided by neural fibers that are recruited by the stimulation, and essentially comprises the sum of the action potentials of recruited neural fibers when they “fire.” U.S. Patent Application Publication 2022/0040486 shows an example where sensing of neural responses is useful in a DBS context, and in particular discusses the sensing of Evoked Resonant Neural Activity, or “ERNA.” It can also be useful to sense other signals in a patient tissue as well, such as stimulation artifacts which results from the electromagnetic field that forms in the tissue as a result of the stimulation, as well as other background signals present in the tissue. See, e.g., U.S. Patent Application Publications 2020/251899 and 2021/0236829. Collectively, a pulse generator can sense an electrospinogram (ESG) signal comprising some or all of these signals.

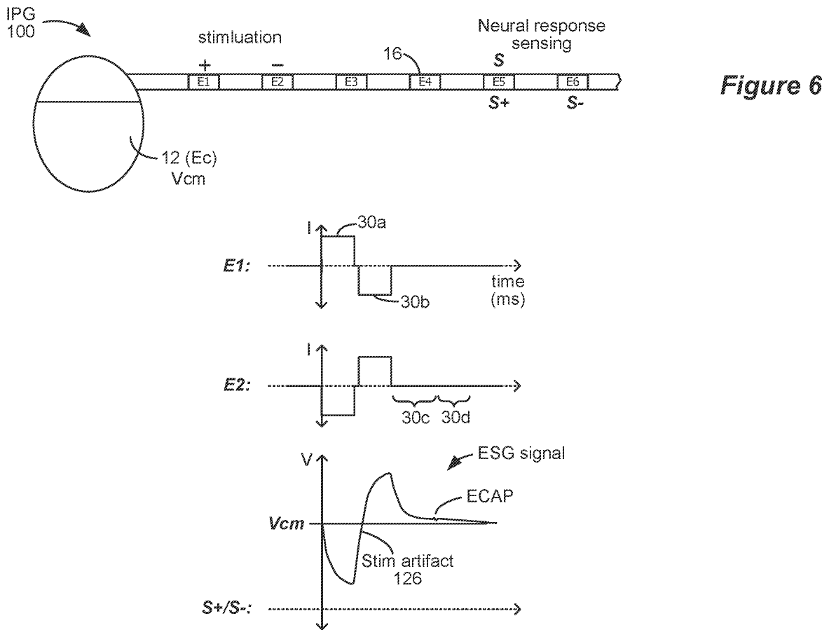

FIG. 5 shows basic circuitry for sensing an ESG signal in an IPG 100. The IPG 100 includes control circuitry 102, which may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 10 as described earlier, which ASIC(s) may additionally include the other circuitry shown in FIG. 5.

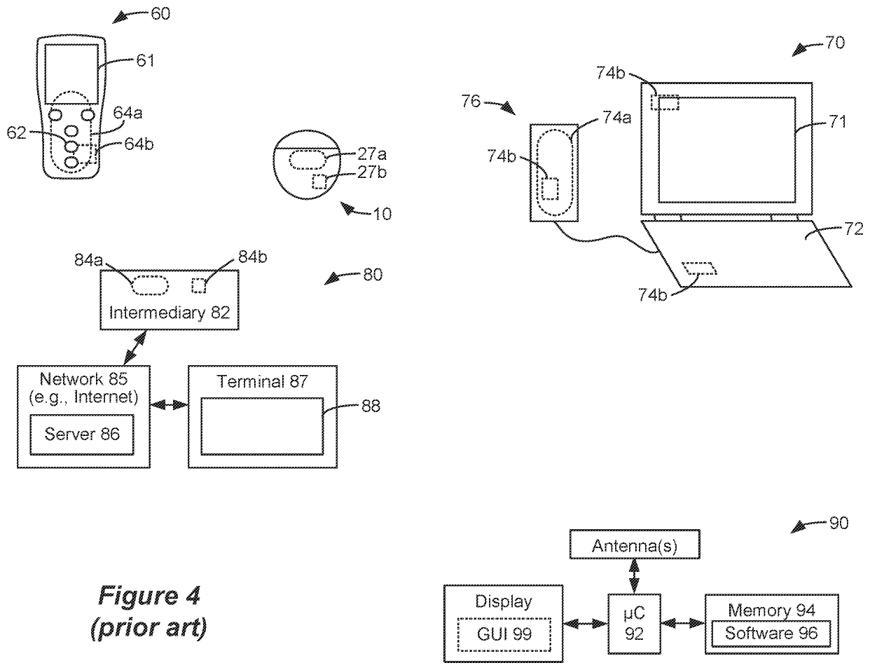

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The electrode current paths to the electrodes 16 include the DC-blocking capacitors 38 described earlier.

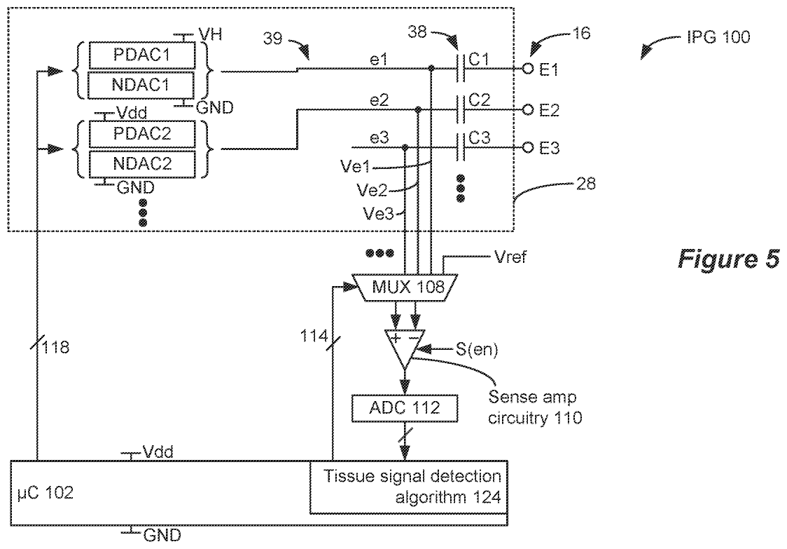

FIG. 5 also shows circuitry used to sense ESG signals. As shown, the electrode nodes 39 are input to a multiplexer (MUX) 108. The MUX 108 is controlled by a bus 114, which operates to select one or more electrode nodes, and hence to designate corresponding electrodes 16 as sensing electrodes. The sensing electrode(s) selected via bus 114 can be determined automatically by control circuitry 102 and/or a tissue signal detection algorithm 124, as described further below. However, the sensing electrode(s) may also be selected by the user (e.g., a clinician) via an external system 60, 70 or 80 (FIG. 4).

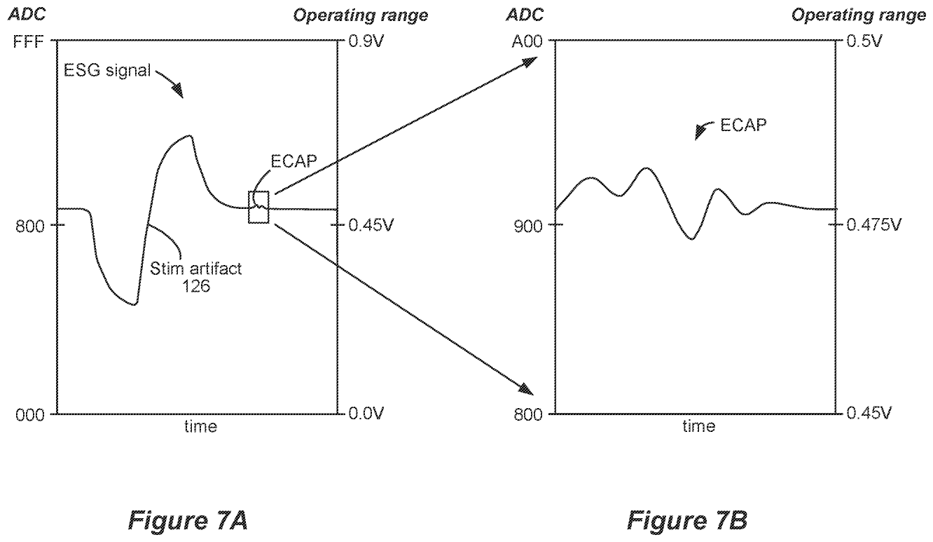

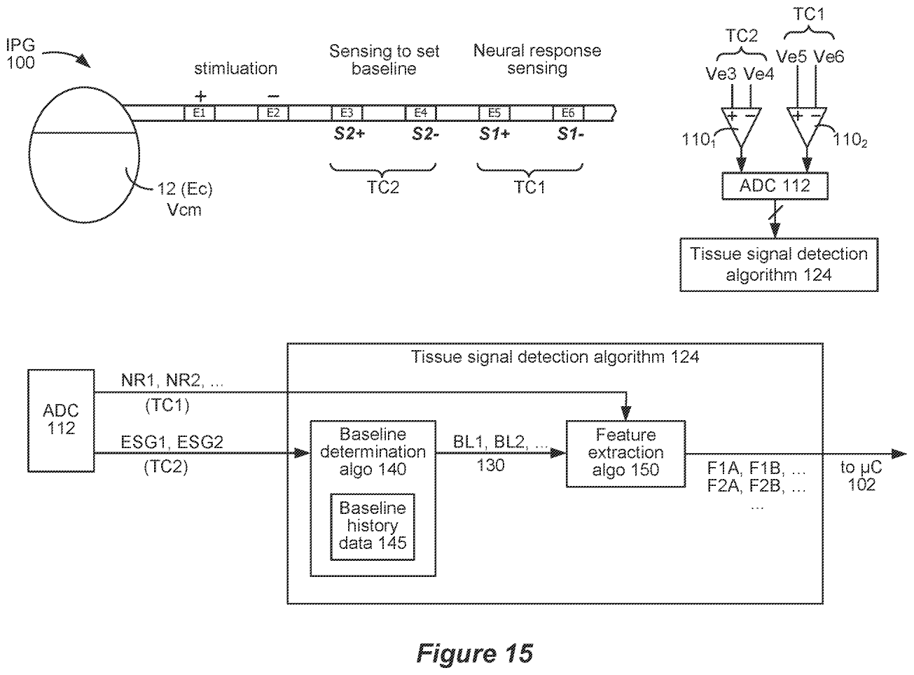

Electrodes selected as sensing electrodes are provided by the MUX 108 to a sense amplifier circuitry 110, and sensing can occur differentially using two sensing electrodes, or using a single sensing electrode. This is shown in the example of FIG. 6. If single-ended sensing is used, a single electrode (e.g., E5) is selected as a single sensing electrode (S) and is provided to the positive terminal of the sense amp circuitry 110, where it is compared to a reference voltage Vref provided to the negative input. The reference voltage Vref can comprise any DC voltage produced within the IPG, such as ground, the voltage of the battery (Vbat), or some fraction of the compliance voltage VH (such as VH/2). If differential sensing is used, two electrodes (e.g., E5 and E6) are selected as sensing electrodes (S+ and S−) by the MUX 108, with one electrode (e.g., E5) provided to the positive terminal of the sense amp circuitry 110, and the other (e.g., E6) provided to the negative terminal. Although only one sense amp circuit 110 is shown in FIG. 5 for simplicity, there could be more than one with each operating in its own timing channel. See, e.g., FIG. 15. The timing at which sensing occurs can be affected by a sensing enable signal S(en). Further details of sense amp circuitry 110 are disclosed in U.S. Patent Application Publication 2023/0173273, which is incorporated by reference in its entirety.

The analog waveform comprising the sensed ESG signal and output by the sense amp circuitry 110 is preferably converted to digital signals by an Analog-to-Digital converter (ADC) 112, and input to the IPG's control circuitry 102. The ADC 112 can be included within the control circuitry 102's input stage as well. The control circuitry 102 can be programmed with a tissue signal detection algorithm 124 to evaluate the digitized signals, such as neural responses, stimulation artifacts, and possibly other signals, and to take appropriate actions as a result. For example, the tissue signal detection algorithm 124 may change the stimulation in accordance with a sensed neural response within the ESG signal (e.g., an ECAP), and can issue new control signals via bus 118 to change operation of the stimulation circuitry 28 to affect better treatment for the patient. The tissue signal detection algorithm 124 may also cause the selection of new sensing electrode(s), which can be affected by issuing new control signals on bus 114. Selecting optimal sensing electrode(s) can be important, and may be determined in light of stimulation that is being provided. In this regard, sensing electrodes (e.g., E5 and E6) may be selected near enough to the electrodes providing stimulation (e.g., E1 and E2) to allow for proper neural response sensing, but far enough from the stimulation that the stimulation doesn't substantially interfere with neural response sensing. See, e.g., U.S. Patent Application Publication 2020/0155019.

Neural responses to stimulation such as ECAPs are typically small-amplitude AC signals on the order of microVolts or milliVolts, which can make sensing difficult. The sense amp circuitry 110 needs to be capable of resolving this small signal, and this is particularly difficult when one realizes that this small signal typically rides on a background voltage otherwise present in the tissue. As explained in U.S. Patent Application Publication 2020/0305744, which is incorporated by reference in its entirety, this background voltage can be caused by the stimulation itself. This is shown in the waveforms at the bottom of FIG. 6, which shows the current stimulation pulses, and the ESG signals received at selected sensing electrodes S+ or S−. The ESG signal includes a neural response—in this case an ECAP—and may also include a stimulation artifact 126 which results from the electromagnetic field that forms in the tissue as a result of the stimulation. The neural response (e.g., ECAP) may be present during active stimulation (e.g., during phases 30a or 30b) when the stimulation artifact is higher and perhaps varying more significantly, or after active stimulation (as shown in FIG. 6) when the stimulation artifact is lower (e.g., during passive charge recovery or quiet periods 30d). Because the DAC circuitry used to provide the stimulation is powered by power supply voltages VH and ground (see FIG. 3), the stimulation artifact 126 will vary between these voltages, and can comprise several Volts.

Differential sensing is useful because it allows the sense amp circuitry 110 to subtract any common mode voltages like the stimulation artifact 126 present in the tissue, hence making the neural response easier to resolve. See, e.g., the above-incorporated '829 Publication. However, this will not remove the stimulation artifact 126 completely, because the stimulation artifact 126 will not be exactly the same at each sensing electrode. Therefore, even when using differential sensing, it may be difficult to resolve the small signal neural response which may still ride on a significant background voltage. U.S. Pat. No. 11,040,202, which is incorporated herein by reference in its entirety, describes circuitry that assists in neural sensing by holding the tissue via a capacitor (such as the DC-blocking caps 38) to a common mode voltage, Vcm. This common mode voltage Vcm is preferably established at the conductive case electrode Ec as shown in FIG. 6, although another lead-based electrode could also be used to provide Vcm. See, e.g., U.S. Patent Application Publication 2023/0138443. As these references disclose, it is beneficial to establish Vcm with reference to the power supply voltage of the DAC circuitry—i.e., the compliance voltage VH explained earlier—because the voltages in the tissue will be between this voltage and ground. Most preferably, Vcm can equal approximately VH/2. In any event, when a common mode voltage Vcm is provided to the tissue, AC signals present in the tissue (neural responses, any stimulation artifacts) will also be referenced to this voltage. This is a helpful improvement, because it tends to stabilize the DC level of the signals being input to the sense amp circuitry 110 by the sensing electrodes.

Sometimes it is useful to sense stimulation artifacts 126 in their own right, because like neural responses they can also provide information relevant to adjusting a patient's stimulation, or to automatically selecting a best combination of sensing electrodes. See, e.g., U.S. Patent Application Publications 2020/251899 and 2021/0236829.

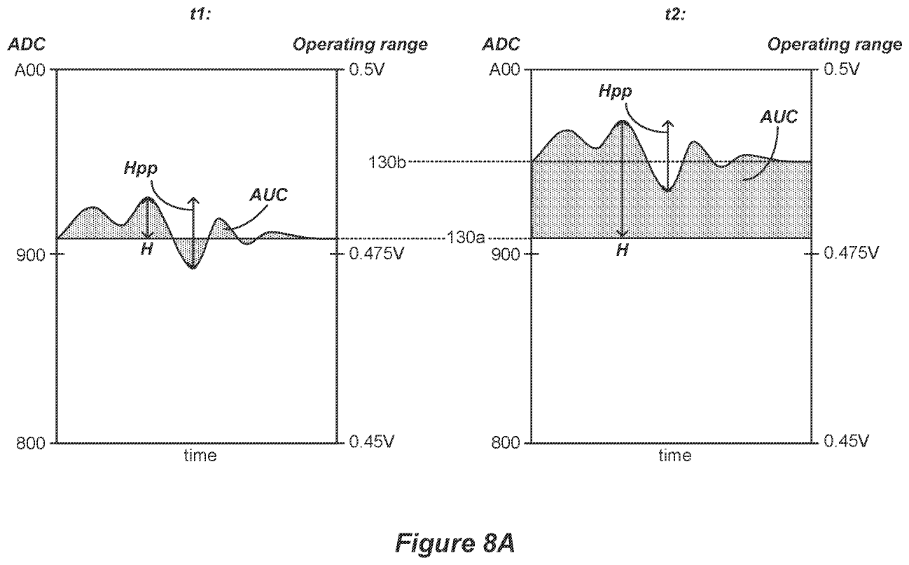

Examples of an ESG signal and a neural response within the ESG signal are shown in FIGS. 7A and 7B respectively after they have been amplified (by sense amp circuitry 110) and digitized (by ADC 112). Once a neural response is digitized, it can be assessed by the tissue signal detection algorithm 124 to determine one or more features of the neural response, which allows the control circuitry 102 to use the values of those features to take appropriate actions as discussed above. As stated for example in U.S. Patent Application Publication 2020/0305744, many features may be determined for the neural response, which may include but are not limited to:
a height of any peak present in the neural response;
a peak-to-peak height between any two peaks (e.g., Hp_p);
a ratio of peak heights;
a peak width of any peak (e.g., a full width half maximum of a particular peak);
an area under any peak;
a total area; this may comprise an area under positive peaks with the area under negative peaks added as discussed further below;
a line length of any portion of the curve of the neural response;
any time defining the duration of at least a portion of the neural response;
a time delay from stimulation to the issuance of the neural response, which is indicative of the neural conduction speed of the neural response, which can be different in different types of neural tissues;
any mathematical combination or function of these or other variables that characterize the neural response.
   These features may be indicative of AC characteristics of the neural response. The tissue signal detection algorithm 124 can also assess other signals in the detected ESG signal, such as the stimulation artifact 126, and may also determine features of such artifacts or other signals.

The ESG signal as digitized in FIG. 7A assigns a digital value to each voltage in the waveform as a function of time in accordance with the ADC 112's sampling rate. In the depicted example, it is assumed that the ADC 112 can digitize voltages within an operating range from 0 to 0.9V. One skilled in the art will understand that the sense amp circuitry 110 can include analog processing circuitry between the output of the amplifier and the ADC 112 to set this operating range, such as attenuators, level shifters, and the like. See, e.g., the above-incorporated '273 Publication. If it is assumed that a 12-bit ADC 112 is used, the ADC 112 assigns sampled voltages with a digital hexadecimal value that can range from 000 (e.g., 0.0V, 000000000000 in binary) to FFF (e.g., 0.9V, 111111111111 in binary), with a digital value of 800 (e.g., 100000000000 in binary) being in the middle (0.45V).

Notice that the smaller-signal neural response as shown specifically in FIG. 7B essentially comprises a smaller piece of the overall sensed ESG signal of FIG. 7A, and as such occurs over a shorter sampling window, and within a smaller range of voltages (e.g., from 0.45 to 0.5 V, or approximately 800 to A00 in hexadecimal). The tissue signal detection algorithm 124 may be able to automatically determine the portion of the sensed ESG signal that includes only the neural response by looking for AC characteristics representative of the neural response. Thus, the IPG 100 can be programmed to sense the ESG signal more broadly (FIG. 7A), or just the neural responses (FIG. 7B) if those are the only signal of interest. In this regard, the timing and duration of the sensing window may be programmable, or the algorithm 124 can be programmed to consider only a desired portion of the data in the sensing window.

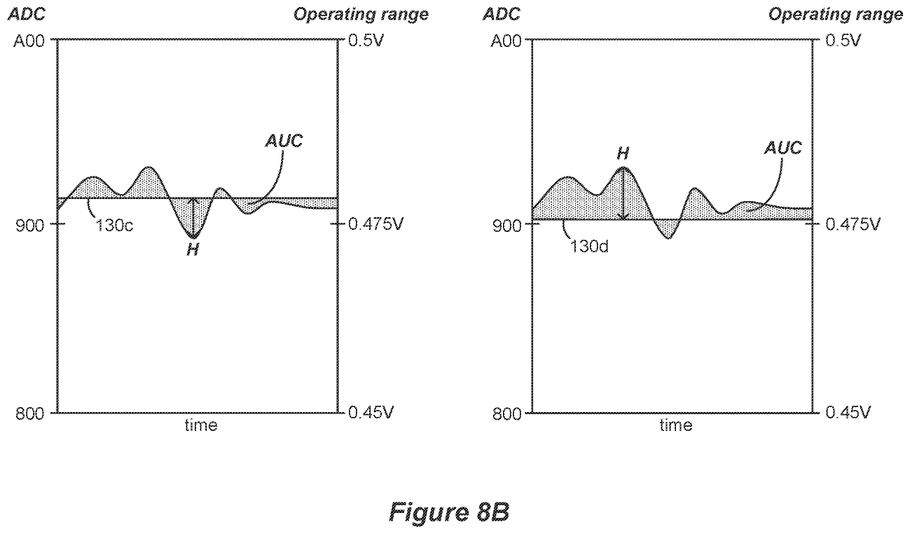

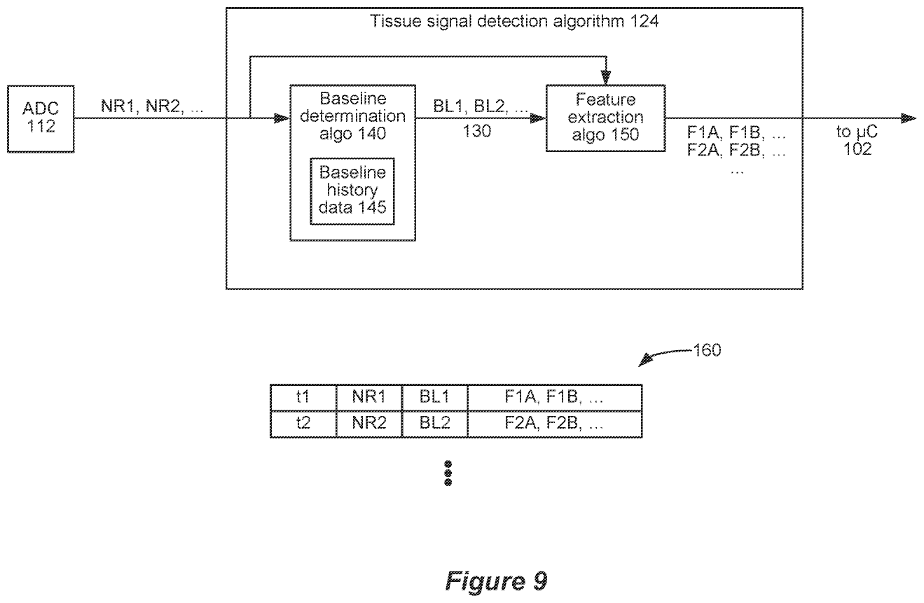

FIG. 8A shows sensed and digitized neural responses during different sensing windows, and two such windows are shown at times t1 and t2 after different stimulation pulses have issued. As shown, the AC signals comprising the neural response may naturally reference to (e.g., be centered around) a particular DC offset voltage. For the waveform shown at time t1, this DC offset voltage is about 0.475 Volts. The value of this DC offset voltage can be affected by many potential factors, such as: the background voltage in the tissue (e.g., as affected by stimulation artifacts 126); the current value of the compliance voltage VH; how the sensed signals are processed by the sense amp circuitry 110 (using attenuators, shifters and the like); the particular electrodes selected for sensing; whether single-ended or differential sensing is used; whether measures are taken to establish a common mode voltage in the tissue, etc. Other biological processes present in the patient may also cause the DC offset voltage to shift at different times, such as respiration and heartbeat. Because some of these factors may vary over time, the DC offset voltage of the neural response may vary over time as well. For example, time t2 shows the sensed waveform later, and it can be seen that the DC offset voltage of the neural response has shifted upwards to about 0.49 Volts.

This change in the DC offset voltage (from t1 to t2) may not be clinically significant because it does not result from an underlying change in the neural response. Instead only the AC aspects of the neural response may be clinically significant. In this regard, the neural responses shown at times t1 and t2—having the same shape (and AC characteristics), but varying only in their DC offset voltages—may be for all intents and purposes be the same, and should be interpreted by the tissue signal detection algorithm 124 as the same.

The tissue signal detection algorithm 124 preferably considers a baseline voltage when determine at least some neural response features, and this baseline voltage may be affected by the DC offset voltage. Suppose for example that the tissue signal detection algorithm 124 uses a baseline 130a to assess and determine neural response features at t1. The algorithm 124 can determine certain neural response features relative to this baseline 130a, like maximum peak height H, or an area under the curve calculation (AUC). (As shown in the shading in FIG. 8A, the absolute value of the area relative to baseline 130a can be determined, in which negative areas below the baseline 130a are attributed positive values. But this is not strictly necessary, and instead these negative areas can be subtracted from the positive areas above the baseline 130a).

If the DC offset of the measured waveform later shifts, as at time t2, using this same baseline voltage 130a may no longer be appropriate. For example, the maximum peak height H and the area under the curve (AUC) now appear much larger at time t2 relative to baseline 130a used at time t1. Keeping the baseline voltage constant without compensating for the change in the DC offset may therefore cause the tissue signal detection algorithm 124 to inadvertently determine that significant changes have occurred in the neural response from t1 to t2, when in reality, the neural response remains unchanged. Instead, it may be more appropriate to determine neural response features at time t2 relative to a baseline 130b, which compensates for the shift in the DC offset voltage. Using 130b as the baseline at t2 would (more accurately) produce the same values for features such as H and AUC as determined at time t1.

(Note that at least some neural response features the tissue signal detection algorithm 124 may determine may not require referencing to a particular baseline voltage. For example, a maximum peak-to-peak height (Hpp) can be determined without reference to a baseline voltage, and notice that these values for Hpp are the same at t1 and t2. The line length of the neural response is another example of a feature that can be determined without use of a baseline voltage).

Establishing an appropriate baseline voltage for assessing features of a neural response is useful even if the DC offset of the digitized waveform does not change, as shown in FIG. 8B. Here, two identical waveforms are shown with the same DC offset, but the baseline voltages 130c and 130d determined by the tissue signal detection algorithm 124 are different. As before, this affects the computation of certain neural response features such as H and AUC. At first glance, it may seem odd that the algorithm 124 may determine the baselines 130c and 130d in FIG. 8B to be different when the waveforms are the same. But in practice, measured neural responses are small, noisy, and not perfectly uniform. Therefore, even if a uniform neural response is measured at different points in time, a unique baseline voltage may need to be determined that is slightly different for each.

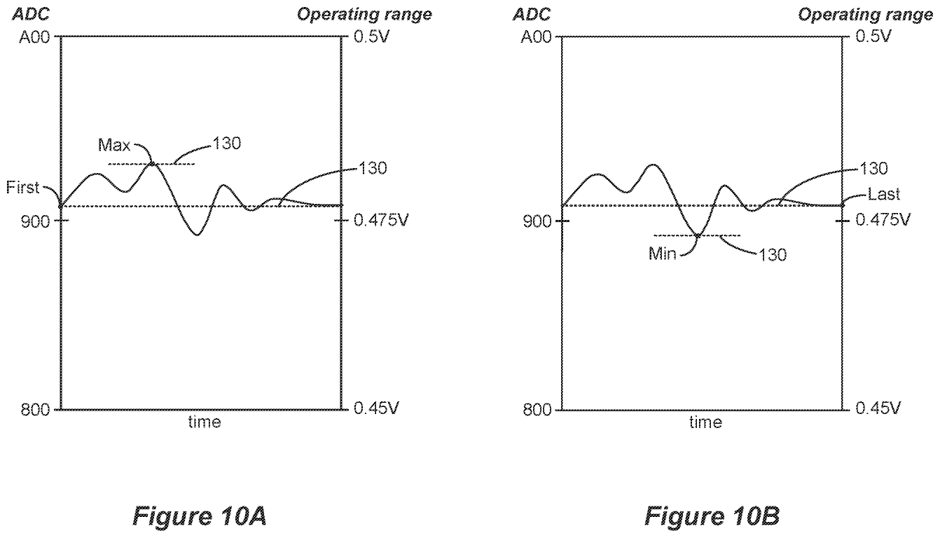

Accordingly, the inventors disclose techniques for determine a baseline voltage for sensed neural responses or other sensed signals in an implantable stimulator device, which allows features of the neural response or other signals to be more easily and reliably established. Preferably, the determined baseline voltage is indicative of a DC offset voltage of the response. An example of an implementation is shown in FIG. 9. This example shows modification to the tissue signal detection algorithm 124 described earlier, which as noted can determine a number of features (H, Hpp, AUC, etc.) for a sensed and digitized neural response or other signals in an ESG signal. In this example, the tissue signal detection algorithm 124 has been programmed to include a baseline determination algorithm 140 for determining a baseline voltage 130 (e.g., BL1) for each sensed neural response (e.g., NR1). As discussed further below, baseline determination algorithm 140 can determine a baseline voltage 130 that compensates for variation in the DC offset voltage based on an analysis of the shape of the neural response, or the shape of the detected ESG signal more generally. Preferably, the determined baseline voltage 130 is represented digitally.

This determined baseline voltage 130 is provided to a feature extraction algorithm 150 which can determine one or more features (e.g., features A, B, etc., or F1A, F1B, etc.) for the neural response (e.g., NR1), with at least some of these features (e.g., maximum peak height H, area under the curve AUC) being determined with respect to the determined baseline voltage 130. (As noted above, the feature extraction algorithm 150 may be able to determine some features (e.g., peak-to-peak height Hpp) without reference to a determined baseline voltage).

Operation of the tissue signal detection algorithm 124 preferably determines a data set 160 which is passed to the control circuitry 102. The data set 160 preferably includes the feature(s) (FiA, FiB, etc.) for different neural responses (NRi) sensed over various sensing windows (ti). The control circuitry 102, as noted above, can then use the determined neural response feature(s) to useful ends, such as to control or adjust the stimulation, select new or different sensing electrodes, monitor stimulation generally, and the like. The control circuitry 102 may process the resulting features before use, such as by averaging them to reduce noise, or the algorithm 124 can do the same before reporting the features to the control circuitry 102.

Optionally, the baseline determination algorithm 140 can consider baseline history data 145 when determining a baseline voltage 130 for a neural response. Baseline history data 145 comprises baseline voltages as previously determined by the baseline determination algorithm 140, which may be used in determining a baseline voltage for a present neural response under review. In one example, the baseline history data 145 comprises at least some of the previously-determined baseline voltages, such as those occurring over a most-recent time interval, or some number of most-recently determined baseline voltages. In one example, the baseline history data 145 can include or compute a moving average of such most-recent baseline voltages.

When determining a baseline voltage 130 for a present neural response using data 145, the algorithm 140 can determine an initial baseline voltage for the neural response based on an analysis of its shape (as discussed in further detail below), but can additionally consider previous baseline voltages stored as part of data 145 before determining a final baseline voltage for that response that will be used during feature extraction (150). For example, the algorithm 140 may average the initially-determined baseline voltage with most-recent baseline voltages stored in data 145 to determine a final baseline voltage to use in assessing the neural response. Such averaging may be weighted to allow algorithm 140 to determine a final baseline voltage that is influenced by the initially-determined baseline voltage, or by previously-determined baseline voltages, to greater or lesser degrees. The rationale to using baseline history data 145 in this fashion relates primarily to noise in the received neural responses, which can distort their shapes, and therefore distort a determination of a baseline voltage based on an analysis of shape. Assessment of historical baseline voltage data reduces noise and variation in the determined baseline voltage 130 on a small time scale. This is sensible, because while a goal of algorithm 140 is to determine an appropriate baseline voltage 130 for neural response assessment to compensate for variation in the DC offset voltage of the sensed ESG signal, such variation typically occurs on a longer time scale than the rate at which baseline voltages and resulting features are determined for the neural responses.

While the tissue signal detection algorithm 124, and its sub-components 140, 145, and 150, have been described as programming (firmware) with programmable logic control circuitry 102, one skilled in the art will understand that other discrete digital or analog circuitry can be used to performed some or all of the described functions of this algorithm 124 or its sub-components.

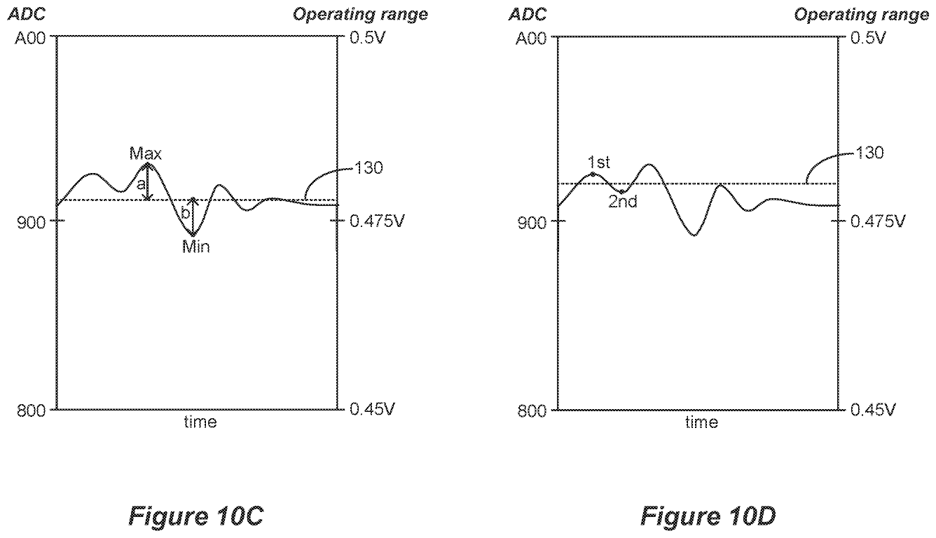

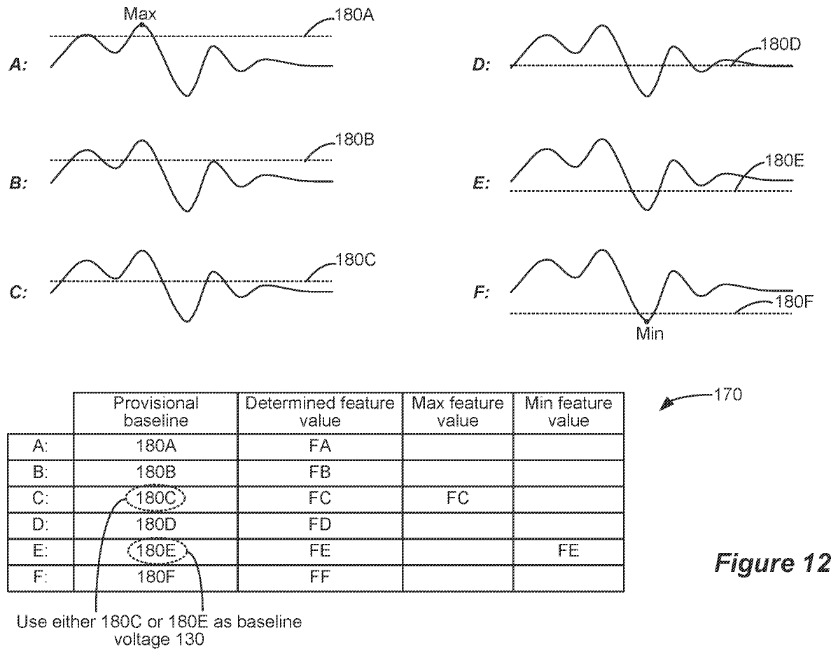

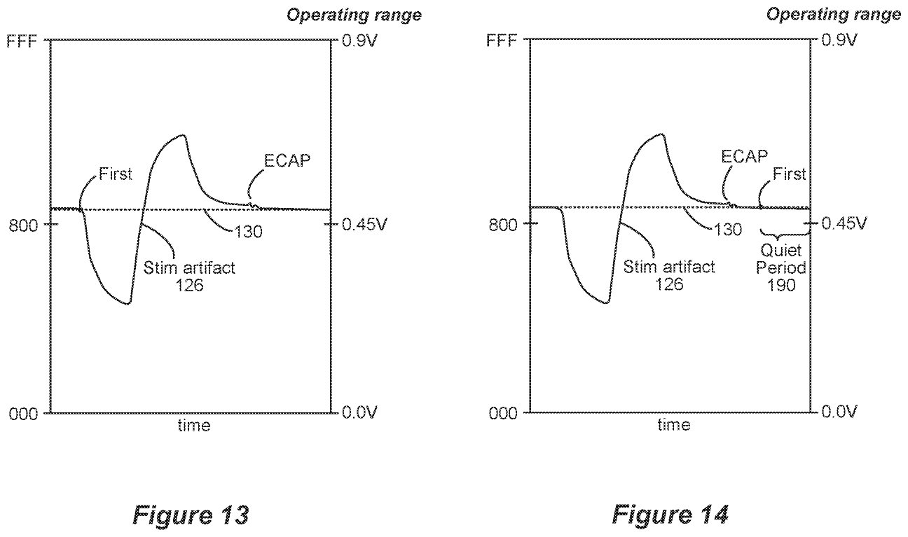

Operation of baseline determination algorithm 140, and manners in which this algorithm 140 can be used to determine a baseline voltage 130, is discussed with reference to FIGS. 10A-14. As noted earlier, algorithm 140 can determine a baseline voltage 130 based on an analysis of the shape of the neural response (or the ESG signal more generally). As such, the baseline voltages 130 in these examples sets the baseline with reference to some aspect of the neural response (or again, the ESG signal). In the examples of FIGS. 10A-12, it is assumed that only the neural response portion of the ESG signal, such as an ECAP, is assessed when determining a baseline voltage 130. FIGS. 13 and 14 differ in that they consider different aspects of the ESG signal—such as the stimulation artifacts or quiet periods in the ESG signal—when determining a baseline voltage. Further, all subsequent examples assume that the baseline voltage 130 is determined based on an analysis of a presently-received ESG signal alone. Thus, baseline history data 145 is not used in determining the baseline voltage 130 in these examples, although as explained such data 145 could also be used in all subsequent examples.

One skilled in the art will notice that the various examples that follow will determine baseline voltages 130 at different absolute values, which would in turn affect the values of at least some of the neural response features (e.g., H, AUC) determined later by the feature extraction algorithm 150. This is fine, so long as the baseline voltage 130 is established consistently. A consistent baseline voltage will allow the control circuitry 102 to determine if there has been a significant change in the AC characteristics of the sensed neural response, as represented by a significant change in the value of the neural response features, and to take appropriate action in response.

In the example of FIG. 10A, the baseline determination algorithm 140 determines the baseline voltage 130 at the voltage value associated with the first point in the sensed neural response signal. FIG. 10B is similar, but determines the baseline voltage 130 at the voltage value associated with the last point in the sensed neural signal. These first and last points, like other points in the neural response, will vary as the DC offset varies, and have the benefit that they are computationally easy to determine. Other easily identifiable points in the neural response can be used to set the baseline voltage 130, such as the maximum voltage value (FIG. 10A) or the minimum voltage value (FIG. 10B) in the neural response. Still other easily identifiable points in the neural response could be used to set the baseline voltage 130, such as the voltage values of various other peaks in the response, although this isn't shown in FIGS. 10A and 10B. The baseline voltage 130 can also be set relative to an identifiable point, such as the minimum voltage value plus an amount, or the maximum voltage value minus an amount, etc.

FIG. 10C determines the baseline voltage 130 with reference to both the maximum and minimum voltage values in the neural response. The baseline 130 in this example is set somewhere between these points in accordance with programmable scalars a and b. Notice that if a=b, the baseline voltage 130 would be set at a voltage value midway between the maximum and minimum values, although this isn't strictly necessary. FIG. 10D shows another example where the baseline voltage 130 is set in accordance with two different peaks in the neural response that are not necessarily at the maximum and minimum values. In this example, the peaks correspond to the first and second peaks in the neural response, although any two peaks (e.g., first and last) could also be used. As with the example of FIG. 10C, the baseline 130 could be set between the two peaks (e.g., using scalars a and b).

The example of FIG. 10E determines the baseline voltage 130 as a point in the neural response having a maximum slope. Mathematically, the maximum slope can be determined by taking the first derivative of the neural response. The maximum slope can be the maximum of the absolute value of the slope, although a maximum positive slope or a maximum negative slope could also be considered. In FIG. 10F, the maximum slope is identified, as well as a local maximum and a local minimum that bound that slope. The baseline voltage 130 is then determined using these local extremes. For example, the baseline voltage 130 can be set between the voltage values of the local maximum and the local minimum, as discussed previously with respect to FIG. 10C.

In FIG. 10G, the baseline voltage 130 is determine as the value corresponding to the maximum curvature of the neural response. Mathematically, this point of largest curvature can be determined by taking the second derivative of the neural response.

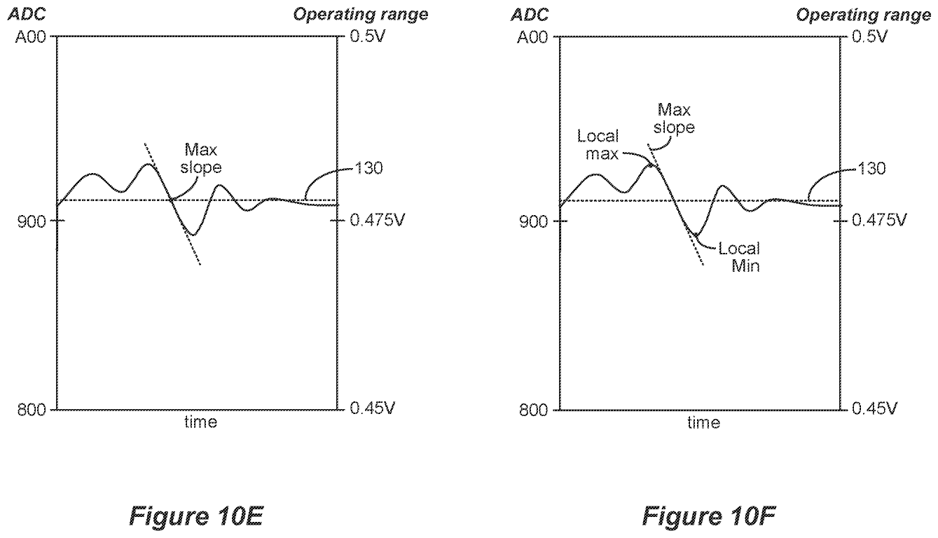

FIG. 11 shows approaches to determine the baseline voltage 130 that involve determining one or more segments 170 in the neural response. Such segments 170 can be determined in a number of different ways, but as shown, the segments 170 are defined as line segments connecting sequential peaks in the neural response. Segments 170 however could comprise other smaller units in the neural response, and thus do not necessarily connect various peaks.

The baseline voltage 130 can be determined from such segments 170 in a number of ways. In the example shown, a longest segment is identified connecting points ‘start’ and ‘end.’ The baseline voltage 130 can then be established using the voltage values of either or both of these end points. For example, and similar to what was shown in FIG. 10C earlier, the baseline voltage 130 can be set at the midpoint values between the values of start and end. The baseline voltage 130 can also be set with reference to only one of these endpoints, in ways described earlier for referencing the baseline voltage 130 to a single point.

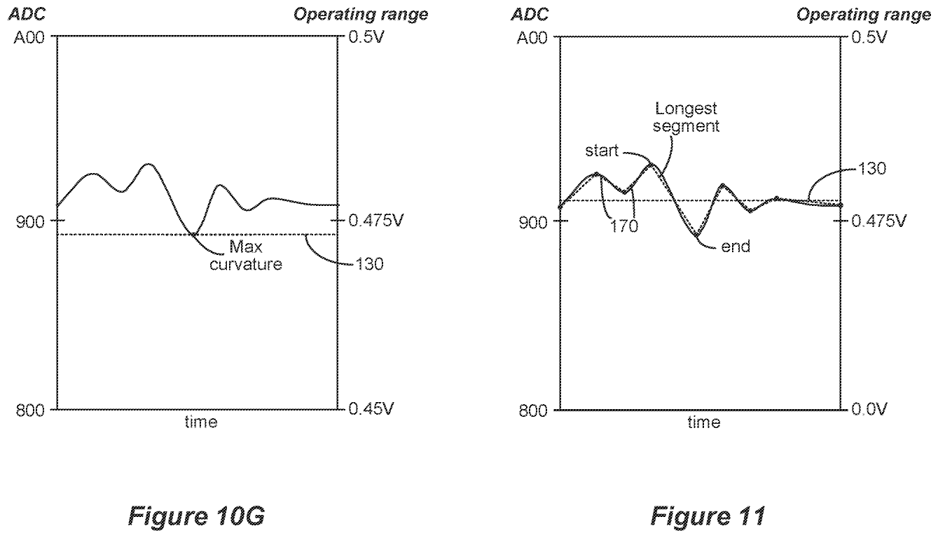

FIG. 12 shows yet another way the baseline determination algorithm 140 can establish a baseline voltage 130 for a neural response. In this approach, the algorithm 140 provisionally tries a number of baseline voltages 180i, and determines a feature (Fi) using that baseline. Preferably, the provisional baseline voltages 180i will sweep though the neural response between maximum and minimum values. Thus, at iteration A, a provisional baseline 180A is tried at or just below the maximum of the neural response, and a feature FA is measured relative to that baseline, as shown in data set 170. At iteration B, the provisional baseline 180A is lowered, and a feature FB is measured relative to that baseline and populated in data set 170. This continues, with the provisional baseline 180F eventually being lowered at iteration F towards the minimum value in the neural response.

Next, the baseline determination algorithm 140 queries data set 170 to inquire which provisional baseline 180i maximizes or minimizes the value of the measured feature Fi. Whether it is useful to a maximum or minimum value for the feature depends on the feature being measured, and user preferences. For example, if the feature of maximum peak height (H) is used, it may be logical to determine the provisional baseline 180i that minimizes this value, as this would correspond to a provisional baseline in the middle in the neural response. If the feature of area under the curve (AUC) is used, it again may be logical to determine the provisional baseline 180i that minimizes this value. However, it may be logical to determine which provisional baseline 180i maximizes a different neural response feature.

Once the minimum (or maximum) of the feature is determined, the baseline voltage 130 is set by the baseline determination algorithm 140 at (or near) the corresponding provisional baseline 180i that minimizes (or maximizes) that feature. For example, and as shown in FIG. 12, feature F is maximized at iteration C (FC) associated with provisional baseline 180C, and is minimized at iteration E (FE) associated with provisional baseline 180E. Thus, the baseline determination algorithm 140 may set the baseline voltage 130 for assessing neural responses at either provisional baseline 180C or 180E.

The baseline determination algorithm 140 may further consider other aspects of a detected ESG signal when setting a baseline voltage 130 for neural response feature extraction. For example, FIG. 13 shows an example in which the stimulation artifact 126 is used to determine the baseline voltage 130. This is useful because the stimulation artifact 126 would, like the neural response, vary with respect to a DC offset voltage.

In this example, the baseline voltage 130 is set at or relative to a first identifiable point in the stimulation artifact 126, akin to what was described earlier in FIG. 10A for the detected neural response. However, the baseline voltage 130 could be set using the stimulation artifact 126 in accordance with other earlier-described techniques, such as: at a last point in the stimulation artifact (e.g., FIG. 10B); relative to maximum and/or minimum values (e.g., FIG. 10C) or to any other peaks (e.g., FIG. 10D) in the stimulation artifact; via identification of a point of highest slope in the stimulation artifact (e.g., FIG. 10E); relative to a local minimum and maximum values bordering a maximum slope in the stimulation artifact (FIG. 10F); via identification of a point of maximum curvature in the stimulation artifact (FIG. 10G); via identification of various segments in the stimulation artifact (FIG. 11); or through determining a baseline that maximizes or minimizes a particular stimulation artifact feature (e.g., FIG. 12). How these other examples would be applied to an assessment of a stimulation artifact 126 should be clear from the descriptions of these earlier examples, and so for simplicity are not shown in FIG. 13.

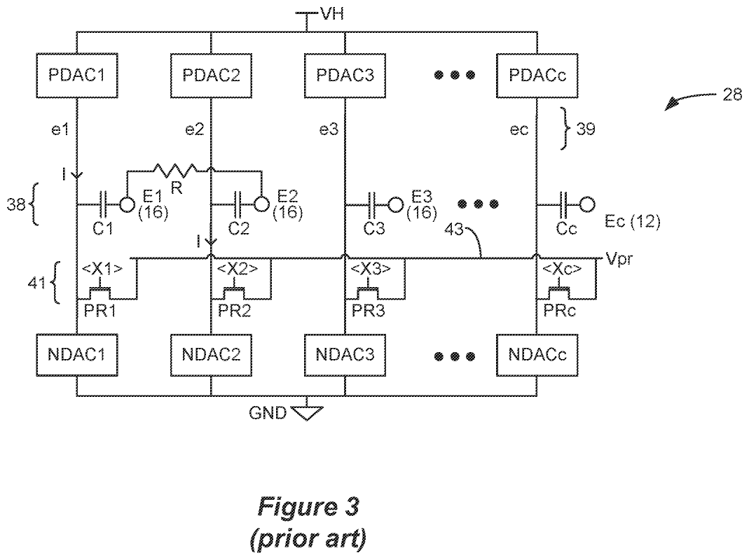

Still other aspects of a detected ESG signal may be used by the baseline determination algorithm 140 when setting the baseline voltage 130 for neural response feature extraction. In FIG. 14, the baseline voltage is set in accordance with a quiet period 190 during ESG sensing in which neither a neural response nor a stimulation artifact 126 are present. This is useful because the quiet period 190 is indicative of the voltage in the tissue to which a DC offset voltage may be referenced. The quiet period 190 used during the baseline voltage determination in FIG. 14 may correspond to the quiet period 30d described earlier (FIG. 2) or may comprise another time period verified by the baseline determination algorithm 140 as devoid of other signals. In the depicted example, the baseline voltage 130 is set at or relative to a first point in during the quiet period 190. However, the baseline voltage 130 could also be set to the last point during the quiet period 190, or at an average or midpoint value during the quiet period. Because the quiet period 190 would not have peaks (as is the case with neural responses and stimulation artifacts), other examples described earlier for determining the baseline voltage 130 that depend on peaks, segments, or that more generally require a changing shape, may not be applicable.

In examples shown to this point, it has been assumed that the baseline determination algorithm 140 determines a baseline voltage 130 in the same timing channel that is used to detect the neural responses. However, this is not strictly necessary, and FIG. 15 shows an alternative in which ESG signals detected in one timing channel (TC2) are used to determine a baseline voltage 130 to assess neural responses sensed in another timing channel (TC1). In the depicted example, neural response are differentially sensed as before at electrodes E5 and E6 in timing channel TC1. A different ESG signal is sensed in a different timing channel TC2 at different electrodes E3 and E4. Sensing in these different timing channels may occur concurrently, or may be time multiplexed. In the example shown, sensing in TC1 occurs using a first sense amp 110₁, while sensing in TC2 occurs using a first sense amp 110₂. The output from each of these sense amps is provided to the ADC 112, which can digitize each output in a time multiplexed manner. Alternatively, each sense amp 110i can output to its own ADC 112 which would allow for simultaneous sensing.

ESG signals as sensed in TC2 are received by the baseline determination algorithm 140, which can determine baseline voltages 130 to be used by the feature extraction algorithm 150 in assessing neural responses (NR) received in TC1. The baseline determination algorithm 140 can determine the baseline voltages 130 in any of the manners previously discussed. Because the ESG signals sensed in TC2 are indicative of the voltage in the tissue to which a DC offset voltage may be referenced, such sensed signals are sensible to use as a reference in determining the baseline voltages.

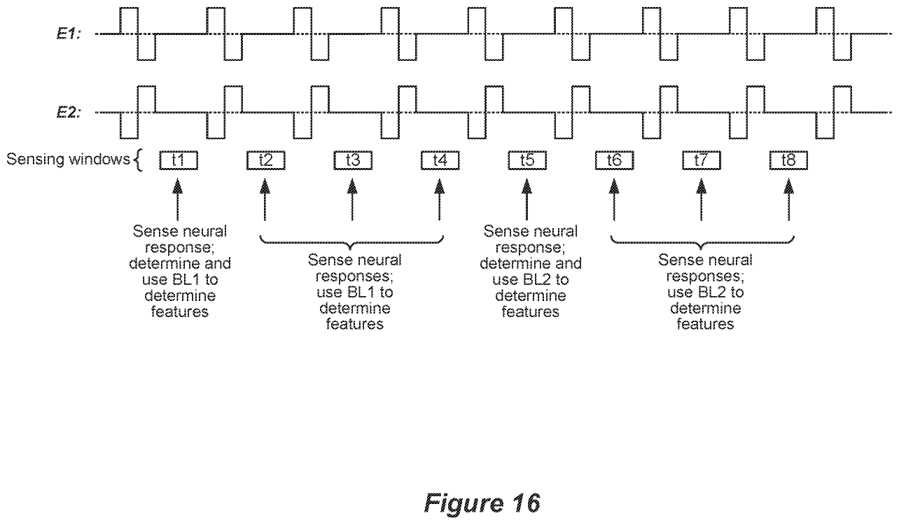

In examples shown to this point, it has been assumed that the baseline determination algorithm 140 determines a unique baseline voltage 130 for each neural response that is sensed. However, this is not strictly necessary, and instead the algorithm 140 may only periodically determine a baseline voltage 130, and use that baseline voltage to assess some number of sensed neural responses that follow. This is shown in FIG. 16. Here, stimulation is provided as pulses at selected electrodes (e.g., E1 and E2). This stimulation may comprise therapeutic stimulation determined to treat a symptom of the patient (e.g., in accordance with a stimulation program determined for the patient), or it may comprise stimulation designed specifically for the purpose of sensing neural responses.

During sensing window t1, a neural response is sensed, and a baseline voltage 130 (BL1) is determined using any of the techniques previously discussed, with BL1 being used to extract one or more features of the neural response. Other neural responses are sensed in timing windows t2-t4, with BL1 as established earlier used to extract one or more features of these neural responses. Thus, a new baseline is not determined in timing windows t2-t4 using the ESG signal carrying the neural response. This process repeats at sensing windows t5-t8. During sensing window t5, a neural response is sensed, and a baseline voltage 130 (BL2) is determined using any of the techniques previously discussed, with BL2 being used to extract one or more features of the neural response. Other neural responses are sensed in timing windows t6-t8, with BL2 as established earlier used to extract one or more features of these neural responses. Essentially, a new baseline voltage 130 is only established for every fourth sensed neural response in this example. Obviously, the number of neural responses for which a determined baseline voltage is used can be varied. This is sensible, because while a goal of algorithm 140 is to determine an appropriate baseline voltage for neural response assessment to compensate for variation in the DC offset voltage of the sensed ESG signal, such variation typically occurs on a longer time scale. It may therefore be unnecessary (and too computationally intensive) to determine a unique baseline voltage 130 to assess each and every neural response that is sensed.

Disclosed examples preferably determine baseline voltages 130 for at least some received neural response, and may determine a baseline voltage for each and every neural response that is received after each stimulation pulse. However, it should be understood that a neural response to stimulation (e.g., NR1) may comprise an average of neural response taken after subsequent pulses.

The various algorithms (e.g., 124, including all or some of its sub-components) and methods disclosed herein can comprise instructions fixed in a computer readable medium, such as a solid-state memory (e.g., control circuitry 102), optical or magnetic disk, and the like. These media may be within the IPG 100, or stored on external systems in manner downloadable to the IPG, such as on various Internet servers (e.g., 86, FIG. 4), manufacturing computer systems, and the like.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for operating a stimulator device comprising a plurality of electrode nodes, wherein each of the electrode nodes is associated with a different electrode configured to contact a patient's tissue, the method comprising:
providing stimulation to the patient's tissue via one or more first of the electrode nodes;
sensing a response to the stimulation at one or more second of the electrode nodes;
determining a baseline voltage from the sensed response; and
determining at least one feature of the response using the baseline voltage, wherein the at least one feature is indicative of an AC characteristic of the response.

2. The method of claim 1, wherein the baseline voltage is determined by assessing a shape of the response.

3. The method of claim 1, wherein the baseline voltage is determined as a first or last voltage value in the response.

4. The method of claim 1, further comprising determining one or more peaks in the response, wherein the baseline voltage is determined relative to a voltage value of at least one of the peaks.

5. The method of claim 4, further comprising determining either or both of a maximum peak or minimum peak in the response, wherein the baseline voltage is determined relative to the voltage value of either or both of the maximum peak and the minimum peak.

6. The method of claim 5, wherein the baseline voltage is determined between the voltage value of the maximum peak and the voltage value of the minimum peak.

7. The method of claim 1, further comprising determining a slope of the response, wherein the baseline voltage is determined relative to a voltage value corresponding to a maximum slope in the response.

8. The method of claim 1, further comprising determining a curvature of the response, wherein the baseline voltage is determined relative to a voltage value corresponding to a maximum curvature in the response.

9. The method of claim 1, further comprising determining one or more segments in the response, wherein the baseline voltage is determined using at least one of the segments.

10. The method of claim 9, further comprising determining a longest of the one or more segments, wherein the baseline voltage is determined relative to at least one voltage value in the longest segment.

11. The method of claim 10, wherein the baseline voltage is determined relative to either or both of a start voltage value and an end voltage value of the longest segment.

12. The method of claim 1, whether the baseline voltage is determined at a voltage value that either maximizes or minimizes a value of the at least one feature.

13. The method of claim 1, wherein the response comprises a stimulation artifact which results from an electromagnetic field that forms in the tissue as a result of the stimulation.

14. The method of claim 1, wherein the response comprises a neural response evoked in the tissue in response to the stimulation.

15. The method of claim 1, wherein the stimulation is provided in a sequence of pulses.

16. The method of claim 15, wherein a response to the stimulation is sensed for each pulse, wherein a unique baseline voltage is determined for each of the responses, and wherein the at least one feature of each response is determined using its baseline voltage.

17. The method of claim 15, wherein a response to the stimulation is sensed for each pulse wherein the baseline voltage is determined for a plurality of the responses, and wherein the at least one feature of the plurality of responses is determined using the baseline voltage.

18. The method of claim 1, further comprising digitizing the sensed response, wherein the baseline voltage is determined using the digitized sensed response.

19. A stimulator device, comprising:
a plurality of electrode nodes, wherein each of the electrode nodes is associated with a different electrode configured to contact a patient's tissue;
stimulation circuitry configured to provide stimulation to the patient's tissue via one or more first of the electrode nodes;
sense amplifier circuitry configured to sense a response to the stimulation at one or more second of the electrode nodes;
control circuitry configured to:
determine a baseline voltage from the sensed response, and
determine at least one feature of the response using the baseline voltage, wherein the at least one feature is indicative of an AC characteristic of the response.

20. A non-transitory computer readable medium comprising instructions executable in a stimulator device comprising a plurality of electrode nodes, wherein each of the electrode nodes is associated with a different electrode configured to contact a patient's tissue, wherein the stimulator device is configured to provide stimulation to the patient's tissue via one or more first of the electrode nodes, wherein the instructions when executed are configured to cause the stimulator device to:
sense a response to the stimulation at one or more second of the electrode nodes;
determine a baseline voltage from the sensed response; and
determine at least one feature of the response using the baseline voltage, wherein the at least one feature is indicative of an AC characteristic of the response.
